(19) United States
(12) Patent Application Publication
(10) Pub. No.: US 2010/0217176 A1
(43) Pub. Date: Aug. 26, 2010

(54) METERED GEL DISPENSER

(76) Inventors: R. Dario Norberto Carrara, Oberwil (CH); Arnaud Grenier, Steinbrunn Ie Haut (FR); Holger Kraus, Buus (CH); Peter L. Sadowski, Woodbury, MN (US); Paul Greenhalgh, Ickleton (GB); Matthew Young, Ickleton (GB); Dominic Reber, Ickleton (GB); Martin Murphy, Ickleton (GB)

Correspondence Address: WINSTON & STRAWN LLP;PATENT DEPARTMENT, 1700 K STREET, N.W., WASHINGTON, DC, 20006, US

(21) Appl. No.: 12/775,053

(22) Filed: May 6, 2010

Related U.S. Application Data
(63) Continuation of application No. PCT/US2008/083196, filed on Nov. 12, 2008.
(60) Provisional application No. 60/987,233, filed on Nov. 12, 2007.

(51) Int. Cl. A61M 35/00; A61N 1/30

(52) U.S. Cl. 604/20; 604/310; 604/311

(57) ABSTRACT

The invention relates to a dermal flowable-composition dispenser that includes a body for retaining a supply of the composition, an application portion having an application surface, and a conduit connecting the supply chamber with the application surface for facilitating the reproducible delivery of a predetermined dose of the composition to the application surface. The application surface includes a concave application surface and a front side having an upper rim enclosing the application surface and forming a depression that has a volume that is greater than or equal to the volume of the predetermine dose. Also, the application surface is configured and dimensioned such it can receive the patient's skin to allow for removal of the predetermined dose of the composition, and wherein the concave application surface is shaped such that the entire application surface contacts the patient's skin when the application surface is pressed thereagainst.

### CROSS-REFERENCE TO RELATED APPLICATIONS

This application is a continuation of International Application PCT/US2008/083196 filed Nov. 12, 2008 which claims the benefit of provisional application 60/987,233 filed Nov. 12, 2007, the entire content of each of which is expressly incorporated herein by reference thereto.

### FIELD OF THE INVENTION

The present invention is directed to a dispenser for dispensing a flowable composition, and more particularly to a dispenser for dispensing a flowable medicament, such as for dermal application.

### BACKGROUND OF THE INVENTION

Dermal administration of medicaments often involves applying the medicament in the form of a cream, ointment, gel or the like. The medicament is generally formulated for topical or transdermal application, and can be absorbed into the skin or to otherwise adhere to the surface of the skin for a predetermined period of time during which the medicament is released from the composition into the skin.

Prior dispensers for dermal compositions, including medicament compositions, have been primarily directed toward medicaments where an accurately controlled dose is not required. Such containers include tubes for containing antibacterial ointments and topical steroids or applicators for deodorants and the like. These containers, however, provide no dosing control, no protection against exposure to the delivered substance by third-party users, and no sealing of the container to protect the substance to be dispensed from exposure to air or other contaminants. For example, U.S. Pat. No. 4,801,052 discloses a metering container designed for cosmetic products that includes an advancement mechanism using a self-threading nut. U.S. Pat. No. 5,725,133 discloses a dispenser for storing a dispensable chemical product such as an underarm composition that includes an elevator mounted for axial movement within the container and a reciprocating mechanism for raising the elevator that also allows a slight retraction of the elevator after it has been advanced. U.S. Pat. No. 6,357,945 discloses a dispenser that provides for single hand use in the actuation of the dispenser by providing a push button actuator to dispense the product above the barrel that contains the substance. US Patent Publication 2007/000946-A1 discloses a dispenser for metered dosing of cream-based medicines comprising a barrel, a base having a threaded rod extending therefrom, a riser having at least one flexible seal which engages the barrel, an applicator cap having apertures therein for spreading dispensed cream onto a user's skin. The user positively knows when a metered amount of cream has been dispensed by tactile and audible feedback. Additionally, U.S. Pat. No. 7,210,870 discloses an applicator for dispensing a product with an angled top surface and a disk that is adapted to contact the angled top surface when the travel of the disk is complete.

More recent developments have lead to compositions for dermal applications that include medicaments where accurate dose administration is an important consideration. International Application Pub. No. WO 2006/005135 relates to a container including a receptacle for holding a substance that may include a drug, and having an opening through which a substance can be dispensed, a domed applicator surface adjacent the opening for spreading a thin layer of the substance on the skin and a closure including a cap with a seal or plug for sealing the receptacle. The seal may be opened or closed by rotation of the collar which interacts with a cam follower surface in order to axially move the seal member. The container described, however, provides no dose control, which is necessary for many drug applications, and further provides very little protection against unintended exposure to the substance by a third-party user of the container.

A device is needed that can improve administration of dermal medicaments and allows accurate dispensed-volume control and safety.

### SUMMARY OF THE INVENTION

An embodiment of the present invention relates to a dermal flowable-composition dispenser. As used herein, the term “dermal” is meant to refer to any application of a substance onto the skin, and can include the application of substances that are intended to be topical or transdermal. “Topical” administration is understood to refer to a composition including an active ingredient, such as a drug or medicament that is meant for delivery to the skin, and thus will remain in the general area of application, while “transdermal” administration is understood to refer to a composition including an active ingredient that is absorbed into the skin such that it enters the bloodstream and is carried to other parts of the body.

The dispenser includes a body defining a supply chamber therein for retaining a supply of the composition; an application portion having a front side with a concave application surface disposed externally and facing away from the supply for holding a predetermined dose of the composition for application to a patient's skin; and a conduit fluidly communicating the supply chamber with the application surface for reproducibly delivering the predetermined dose of the composition to the application surface from the supply chamber.

The supply chamber can be integrally formed with the body or may be a separate unit that is inserted into the body. The conduit is preferably disposed generally centrally in the concave application surface. In a preferred embodiment, the application surface is configured and dimensioned such it can receive the patient's skin to allow for removal of the predetermined dose of the composition. Further the application surface can be shaped such that a portion of the skin of a patient contacts substantially the whole application surface when the application surface is pressed against the skin. The application portion can further include a secondary application that is convex in shape and substantially surrounds the application surface.

In a preferred embodiment, the application portion comprises an applicator that is removably affixed to the body with the conduit in said fluid communication. The applicator preferably includes an outwardly-projecting flange disposed near an outer edge of the applicator configured to assist a user of the dispenser in removing the applicator from the body without contacting the application surface by receiving a user's finger therebeneath to remove the applicator from the body. The body can include a concave upper surface that is complementary with the applicator for supporting the application surface. Further, the conduit includes a nozzle, and the applicator comprises a sealing extension extending away from the front surface and defining an opening configured to receive the nozzle in fluid-tight association therewith to retain the composition delivered from the nozzle on the front side. The extension preferably includes an inwardly-turned portion configured and disposed for abutting and sealing against the nozzle and configured for wiping the composition from the nozzle when the applicator is removed from the body.

A further embodiment of the present invention relates to an applicator for use with a dermal flowable-composition dispenser. The applicator includes a front side with a concave application surface disposed externally and being sufficiently large to receive a user's skin for application of the composition thereto, an opening formed through the applicator in communication with the application surface, and an attachment portion for securely engaging the body to releasably retain the applicator on the flowable-composition dispenser.

Another embodiment of the dispenser according to the present invention can include an advancement mechanism with a piston slidably arranged within the supply chamber and having an aperture formed therethrough. A rod passes through the aperture and is arranged such that rotation of the rod causes linear motion of the piston along the central axis of the cavity. A base is affixed to the rod such that a user can cause advancement of the piston by rotating the base.

The advancement mechanism is preferably arranged such that a predetermined dose of the composition is supplied through the opening upon rotation of the base through an angle that is about equal to a predetermined factor of 360°. The body and the base preferably have substantially matching elongate cross sections such that turning the base relative to the body through the predetermined angle causes the cross section of the base to align with the cross section of the body upon rotation of the base through the predetermined angle.

A locking member is preferably disposed between the body and the base such that motion of the base member relative to the body is restricted when the locking member is in an active state, and wherein the locking member is free to be rotated in an advancing direction when the locking member is in an inactive state. The locking mechanism can further include a timer circuit and an actuator controlled by the timer, wherein the timer circuit causes the actuator to prevent the locking mechanism from changing to the inactive state for a predetermined period of time after the base member is rotated through a predetermined closing angle.

In an embodiment, the aperture of the piston can include a self-threading nut configured to form threads in the rod as it is turned, which are used by the nut to cause advancement thereof along the rod. Preferably, the self-threading nut includes a cylindrical interior surface having two start threads formed therein on opposing sides thereof. The start threads are each preferably configured for forming a discrete thread set on the rod.

In an embodiment, the rod can be moveably affixed to the base such that the rod is moveable with respect to the base in a direction parallel to the central axis of the supply chamber. A portion of the rod preferably interacts with a corresponding portion of the body to cause the rod to reciprocate along the direction of movement during rotation of the base through the predetermined angle.

The device can have a one-way, self-sealing valve arranged in said opening. The valve is configured to permit the dose of the composition to be expelled through the opening, otherwise keeping the opening substantially sealed. The self-sealing valve preferably creates an air tight seal with said opening. The valve preferably includes a resiliently deformable member secured to the opening such that pressure of the composition against the resiliently deformable member causes deformation thereof sufficient to allow the composition to be expelled through the opening and such that, upon release of pressure against the resiliently deformable member, the resiliently deformable member returns to a sealing state.

A preferred method for applying a predetermined dose of a composition to the skin of a patient includes the step of affixing an applicator to a body, the body defining a cavity therein for retaining the composition and including an outlet open to the cavity, and the applicator including an application surface facing substantially away from the body when affixed thereto and being substantially concave, and including an opening aligned with the outlet of the body when the applicator is affixed thereto. The method further includes causing the predetermined dose of the composition to be expelled from the cavity such that the dose passes through the outlet and the opening and collects on the application surface. The application surface is placed into contact with the skin of the patient such that the skin contacts substantially the whole application surface such that the dose of the composition within the central concave portion is substantially transferred to the skin of the patient. Also, the applicator is preferably removed from the body, and a second applicator is affixed to the body. The second applicator is preferably similar to the first, and includes an application surface facing substantially away from the body when affixed thereto and being substantially concave, and includes an opening aligned with the outlet of the body when the applicator is affixed thereto. A predetermined dose of the composition is expelled from the cavity such that the dose passes through the outlet and the opening and collects on the application surface of the second applicator. The outlet is preferably configured so as to interact with the application surface such that upon completion of a dose application, the outlet has a minimal amount of the composition remaining on it.

### BRIEF DESCRIPTION OF THE DRAWINGS

FIG. 1 is a perspective view of a dispenser constructed according to an embodiment of the present invention;

FIG. 2 is a cross-sectional view of the dispenser shown in FIG. 1;

FIG. 3 is a top view of a self-threading nut that can be used in an embodiment of the dispenser of FIG. 1;

FIG. 4 is an assembly view showing a locking mechanism included in the dispenser of FIG. 1;

FIG. 5A is a cross-sectional view of a piston reciprocating mechanism that can be used in an embodiment of the dispenser of FIG. 1;

FIG. 5B is a graphical representation of the profiles of cam surfaces included in the mechanism of FIG. 5A;

FIG. 6 is a cross-sectional view showing the detail of a valve included in the dispenser of FIG. 1;

FIG. 7 is a cross-sectional view showing the valve of FIG. 6 during operation thereof;

FIG. 8 is a cross-sectional view showing the detail of an alternative valve structure;

FIG. 9 is a perspective view of the valve structure of FIG. 8;

FIG. 10 is a cross-sectional view of a further alternative valve structure;

FIG. 11 is a cross-sectional view of a further alternative locking mechanism;

FIG. 12 is a cross-sectional view of a dispenser including an advancement mechanism having a motor according to an embodiment of the present invention;

FIG. 13 is a cross-sectional view of a valve similar to that depicted in FIGS. 1 and 7 further including a heating element;

FIG. 14 is a perspective view of a further embodiment of a dispenser including an applicator removal mechanism;

FIG. 15 is a cross-sectional view of a further alternative valve structure; and

FIGS. 16-18 are perspective views of an alternative embodiment of a locking mechanism.

### DETAILED DESCRIPTION OF THE PREFERRED EMBODIMENTS

The invention relates to a dermal flowable-composition dispenser that includes a body defining a supply chamber therein for retaining a supply of the composition; an application portion having an application surface; and a conduit connecting the supply chamber with the application surface for facilitating the reproducible delivery of a predetermined dose of the composition from the supply chamber to the application surface. The application portion has a front side and is disposed externally and facing away from the supply chamber for holding the predetermined dose of the composition for application to a patient's skin. The application surface includes a concave application surface and a front side having an upper rim enclosing the application surface and forming a depression that has a volume that is greater than or equal to the volume of the predetermine dose. The application surface is configured and dimensioned such it can receive the patient's skin to allow for removal of the predetermined dose of the composition, and wherein the concave application surface is shaped such that the entire application surface contacts the patient's skin when the application surface is pressed thereagainst. Preferably, the rim is substantially planar and the application surface has a surface area of between 5 cm² and 15 cm² with a maximum depth of between 1 and 7 mm.

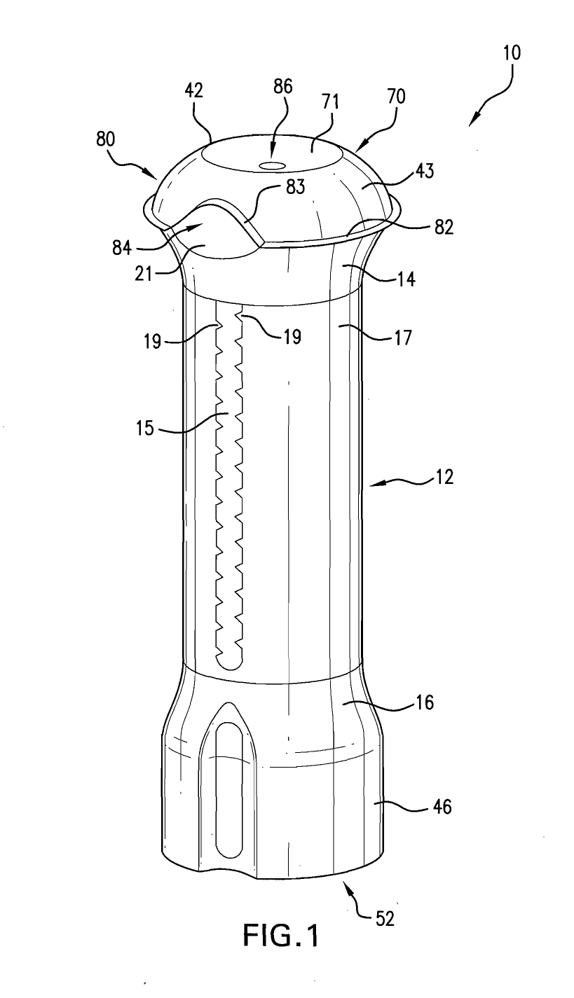

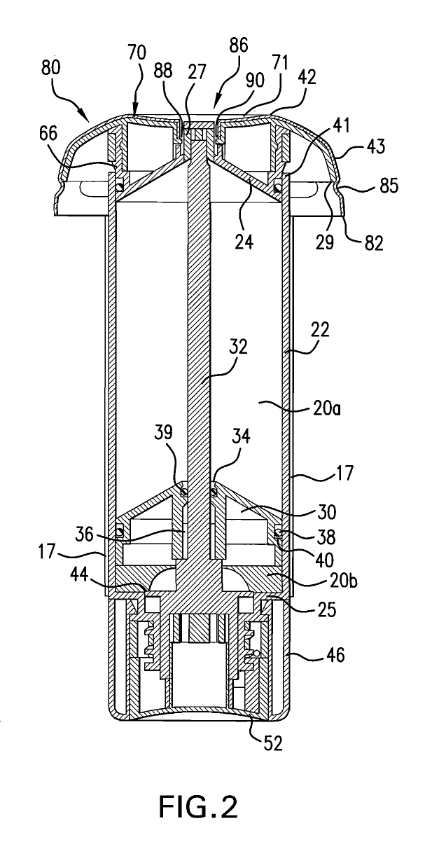

Referring to the figures, where like reference numerals indicate similar features, a dispenser 10 for applying a composition to the skin of a patient according to an embodiment of the present invention is shown in FIG. 1. Dispenser 10 includes a body 12, a proximal end 16 and a distal end 14. As used herein the terms “proximal” and “distal” are made with reference to a third-party user of the device, as the device is to be held in the user's hand. Further, as stated previously, the term “dermal,” as used herein, is meant to refer to any application of a substance onto the skin, and can include the application of substances that are intended to be topical or transdermal. “Topical” administration is understood to refer to a composition including an active ingredient, such as a drug or medicament that is meant for delivery to the skin, and thus will remain in the general area of application, while “transdermal” administration is understood to refer to a composition including an active ingredient that is absorbed into the skin such that it enters the bloodstream and is carried to other parts of the body. Body 12, shown in cross-section in FIG. 2, includes a supply chamber 20 formed therein that is defined by side wall 22, which circumferentially surrounds supply chamber 20, as well as distal wall 24 and proximal wall 25. Distal wall 24 includes conduit 26 formed therein, which allows for fluid communication between supply chamber 20 and distal surface 70 of body 12. Piston 30 is slidably fitted within supply chamber 20 so as to abut the interior surface of side wall 22 around the circumference thereof.

Supply chamber 20 is configured to contain a composition therein. The composition is preferably in the form of a viscous liquid or semi-solid material that contains a medicament that is to be applied to the skin. More preferably, the composition hydraulically behaves like a gel. A semi-solid material, while technically a solid, shares some properties of liquids, such as shape conformity and the ability to flow when under pressure. Preferably, the composition is in the form of a gel, cream, or another type of material that is capable of dermal delivery of a medicament. The composition can also include pastes and liquid/solid mixes. Body 12 is preferably made from a material that is suitable for contact with a gel composition, such as polypropylene (“PP”). It has been determined that a container of PP having a wall thickness of as little as 0.3 mm provides proper barrier for ethanol evaporation from a gel compound. In order to provide adequate structure for body 12, the wall thickness thereof is preferably about 1 to 2 mm. Piston 30 is also preferably made of PP and preferably has a thickness of about 1 to 2 mm, except as otherwise required by the design thereof. Cyclic olefin copolymer (“COC”) can also be used to form body 12 or piston 30, and provides improved barrier properties, but is generally costly.

Supply chamber 20 preferably has a volume of between about 30 mL and 500 mL and preferably between 50 and 200 mL. One embodiment has a volume of about 100 mL, and its supply chamber 20 contains either 60 doses of the composition in an amount of 1.5 mL per dose or 30 doses of the composition at an amount of 3.0 mL per dose. The device can be configured to provide a month's supply of doses.

Depending on the aspect ratio of the circumference of dispenser 10 to the height of dispenser 10, the sensitivity of the dose can be stated comparatively as a percentage of the dose, for example a 1.5 mL dose, as described above in connection with a container including 60 such doses. This comparison assumes that the plunger position is accurate to within 0.1 mm and, thus, does not substantially affect dose sensitivity, particularly with respect to the relatively large 1.5 mL dose of the present example. The accuracy of dose delivery preferably has less than about a 20% tolerance, more preferably less than about a 10% tolerance, and can be around 1% to 7% in some embodiments. A dispenser with circumference between about 100 and 150 mm with a corresponding height of between 135 mm and 90 mm has been found to meet this criteria, assuming use of a mechanism for advancing piston 30 that can control piston position to within about 0.1 mm. Such circumferences generally correspond to a piston 30 having an upper surface with an area of between about 10 cm² and 12 cm². The aspect ratio of circumference to height may also vary with the size of the dose to be administered. Accordingly, when decreasing dose volume, it may be preferable to use a container with a smaller circumference, rather than decreasing the distance through which piston 30 travels during dose administration. For example, a dispenser that is to administer a dose of 3.0 mL may have a circumference that is larger than a dispenser that is to administer a dose of 1.5 mL by a factor of about 1.4, the two dispensers both administering the proscribed dose by moving piston 30 through the same distance. Preferably, the circumference is selected to dispense the desired dose by advancing piston 30 by at least 1 mm, and more preferably at least 1.2 mm. In one embodiment the desired dose can be dispensed by advancing piston 30 by about 5 mm. Preferably, the desired dose is dispensed by advancing the piston a distance between 1.5 and 3.0 mm, and more preferably about 1.6 mm.

Piston 30 is configured to divide supply chamber into an active volume 20a and a dead volume 20b, the active volume 20a containing the composition, and the dead volume 20b preferably being substantially void of the composition. Piston 30 is further configured to vary the ratio of active volume 20a to dead volume 20b within supply chamber 20 by axial movement within supply chamber. A decrease in the volume of active volume 20a by distal sliding of piston 30 within supply chamber 20 increases the pressure of the composition within supply chamber 20, which in turn causes expulsion of an amount of the composition from supply chamber 20 through conduit 26. The expulsion of the composition through conduit 26 continues until the pressure of the composition returns to an equilibrium value, which generally occurs when the amount of the composition expelled through conduit 26 is equal to the reduction in the volume of active volume 20a caused by advancement of piston 30.

To achieve advancement of piston 30 within supply chamber 20, an advancement mechanism is provided that includes a rod 32 that passes through aperture 34 formed through piston 30. Nut 36 is secured within aperture 34 and engages rod 32 such that rotation of rod 32 causes linear motion of piston 30. Preferably, nut 36 is of the typical left-hand configuration whereby clockwise rotation of rod 32 causes motion of piston 30 in a distal direction, and is preferably self threading, such as that which is described in U.S. Pat. No. 4,801,052. Preferably rod 32 is made from PP and is in the form of an unthreaded cylinder. Nut 36 is, accordingly, preferably configured to form threads, such as by cutting, in rod 32 that mate with threads included in nut 36 for advancement thereof. Such an arrangement provides improved sealing of the interface between piston 30 and rod 32 over an arrangement with a pre-threaded rod. Improved sealing can reduce or prevent ingress of oxygen, which can be harmful to some compounds, or other contaminants; leaking of the composition; loss of pressure within the active volume; and evaporation of volatile compounds within the composition. To further enhance the sealing of the interface between rod 32 and piston 30, a seal 39 can be affixed within aperture 34 proximally of nut 36 between piston 30 and rod 32. Seal 39 is preferably formed from a material that is chemically resistant and is compatible with the compositions. Seal 39 can be an o-ring seated within a channel. Suitable materials for such a seal 39 include nitrile rubber, ethylene propylene diene monomer rubber (“EPDM”), silicon Viton, thermoplastic elastomer plastic resin (“TPE”) or the like. In an alternative embodiment, a nut on a prethreaded rod can be provided.

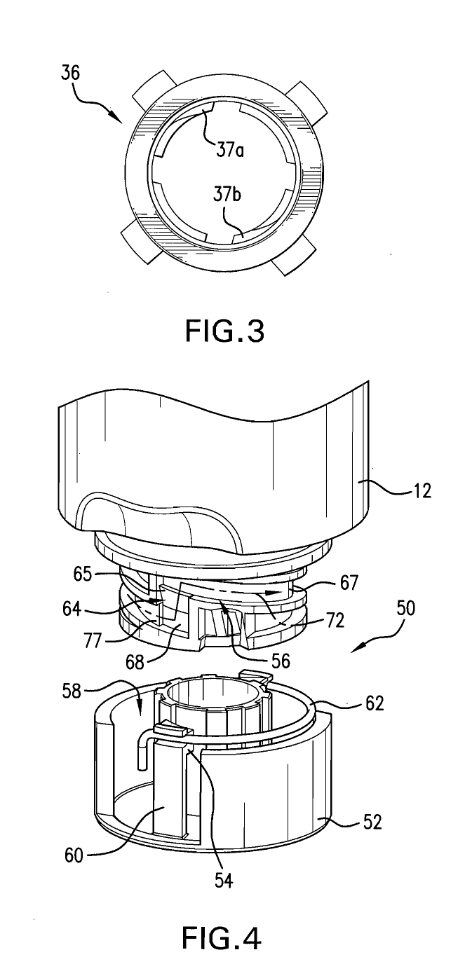

In a preferred embodiment, nut 36 is a twin-start self-threading nut, as shown in FIG. 3A. In such a configuration, nut 36 has two start threads 37a, 37b formed therein such that two discrete thread sets are formed in rod 32 as nut 36 is advanced thereon. Such an arrangement can be advantageous when used in dispenser 10 because the height of the nut 36 can be reduced by as much as 50% while maintaining the same number of threads engaged with the rod 34, which can lead to an overall reduction in the size of dispenser 10. Further, the use of twin starts 37a, 37b provides more even balancing of the forces applied to both piston 20 and rod 32 during use thereof in both the circumferential direction and the axial direction by providing thread support on both sides of rod 32 and by providing cutting force on both sides of rod 32 during advancement of nut 36 thereon.

Sealing of supply chamber 20 is further augmented by providing a seal around the perimeter of piston 30. Preferably, the seal feature is in the form of an o-ring 38, for example formed from EPDM that is seated within a channel 40 formed in the perimeter of piston 30. Other acceptable materials for o-ring 38 can include silicon rubber, nitrile rubber, Viton or other similar materials. Alternatively, the sealing feature can be in the form of a lip seal comprised of a thin flange projecting from the perimeter of piston 30 formed integrally therewith. Further, piston 30 can have a plurality of sealing features of the same of different types. An additional seal can be provided between rod 32 and hole 44 formed in proximal wall 25 of body 12, through which rod 32 passes.

Base 46 is affixed to rod 32 and is used to facilitate turning of rod 32 to cause advancement of piston 30. In an embodiment, base 46 is integrally formed with rod 32 from PP; however, in a preferred embodiment, rod 32 is formed separately from base 46 so that piston 30 can be assembled onto the proximal end of rod 32 prior to assembly of rod 32 onto base 46. Base 46 provides an enlarged feature that assists in turning rod 32 by increasing leverage and providing an enlarged gripping surface for a user of dispenser 10. Preferably, base 46 has a shape that encourages proper dosing of the composition held within dispenser 10. Preferably, the base 46 and body 12 have matching lateral profiles at least in the area where base 46 and body 12 meet. The profile selected should be such that the profiles of base 46 and body 12 are aligned at the beginning and end of the dosing operation. In the embodiment shown in FIG. 1, the shape of base 46 and body 12 is an oval, such that the profiles are aligned at every 180° interval of turning of base 46. When the user begins to turn base 46 relative to body 12, the profiles become un-aligned and remain as such until a 180° turn of base 46 has been completed. This arrangement gives visual and tactile feedback regarding dosage completion and encourages the user to dispense a full dose. In the 180° alignment arrangement, the advancement mechanism is preferably configured to dispense a predetermined amount of the composition by turning the base 46 through an angle of 180°. Thus, the pitch of the threads of nut 36 should provide for proper advancement of piston 30 within supply chamber 20 through the dosing rotation of 180° to dispense the desired dosage or desired fraction thereof. The required travel distance of piston 30 is a function of the cross-sectional area of the supply chamber and varies inversely therewith. In a preferred embodiment, turning the base 46 through an angle of 180° results in the base 46 and the body 12 being substantially aligned and dispensing of approximately 1.5 mL of the composition. The desired dose will depend, however, on the concentration of the medicament within the composition and the amount of medicament to be administered. It is noted that, in this preferred embodiment, a dosage of 3.0 mL of the composition can be administered by turning the base through two successive 180° rotations.

Additional configurations for the matching lateral profiles of base 46 and body 12 are possible. The alignment principle described above can apply to profiles that align through an angle of rotation that is a factor of 360°. The 180° alignment arrangement discussed above fits within this principle, as would a triangular profile that would align at rotations of 120°, for example, as would a squared shape that would align at every 90°, and so forth. Additionally, a non-circular profile, such as a circle with a notch formed therein so as to align every 360°, can be used. The parameters discussed above affecting the amount of the composition expelled per dosing operation would be adjusted to achieve proper dosing through the desired alignment angle. The angular rotation to administer a desired dosage is preferably large enough to keep tolerances for the advancement mechanism at a manageable level to retain a desired quality of visual and tactile feedback.

Such non-circular configurations, particularly with respect to body 12, further serve to prevent piston 30 from rotating within supply chamber 20 during turning of rod 32. Turning of piston 30 within supply chamber 20 can be detrimental to dosing, particularly with predetermined dose administration achieved through a proscribed dosing action, as described herein. Accordingly, supply chamber 20 preferably has a lateral profile around wall 22 that substantially matches the non-circular lateral profile of the outside of body 12, and piston 30 has a lateral profile that matches and slidably fits within supply chamber 20. A circular configuration for supply chamber 20 is possible, however, provided that the balance of axial and rotational forces are appropriate for the relative frictional forces resisting axial and rotational movement within the system.

In a preferred embodiment, dispenser 10 includes a locking mechanism that restricts relative motion between base 46 and body 12. The preferred locking mechanism is configured to include an active state, wherein rotation between the base 46 and body 12 is prevented, to an inactive state, wherein the base 46 can be rotated relative to the body 12. The preferred locking mechanism requires an additional action to be carried out by the user of the dispenser 10 to carry out the dosing action, such as twisting dosing-action discussed above. The locking function of the locking mechanism is preferably configured to prevent accidental or unintentional dosing and discourages accidental double-dosing.

In the embodiment shown in FIGS. 2 and 4, the locking mechanism 50 includes at a pushbutton 52 disposed within base 46. Pushbutton 52 is affixed to base 46 so as to be axially moveable with respect thereto, but rotatably restricted thereto. That is, pushbutton 52 can be depressed with respect to base by pushing upward thereon, but pushbutton 52 and base 46 are not rotatable with respect to each other such that when base 46 is rotated during the dosing operation of device 10, pushbutton is rotated as well. In addition to interacting thusly with body 12, pushbutton includes a pair of cam followers 54 that interact with a cam track 56 formed in a portion of body 12 that extends into base 46 and more particularly into an internal portion 58 of button 52. Cam followers 54 are formed at the end of arms 60 and are urged in a radially inward direction by spring 62.

Cam track 56 includes two distinct sections: an actuation portion 64 and a return portion 67. Cam followers 54 are configured such that they are restricted in motion along a specific path 72 proscribed by cam track 56, including in each of actuation portion 64 and return portion 67. In the arrangement shown, in which button 52 has two cam followers 54, cam track 56 includes two actuation portions 64 and two return portions 67 that are similar in shape and are oppositely-disposed on body 12 and are interconnected so as to form a continuous path.

In an initial position for locking mechanism 50, cam followers 54 are respectively positioned at the lower end 68 of actuation portion 64. The orientation of cam track 56 is such that cam follower 54 is prevented from moving in a lateral direction with respect to body 12 and is only permitted to move vertically. This means that pushbutton 52, and thus base 46 cannot be twisted relative to body 12, which prevents dosing of device 10 as well as backwards movement of the dosing mechanism. The only movement of pushbutton 52 permitted in this position is to depress pushbutton 52 into base 46, which causes cam followers 54 to move over ramps 65 and into the leading end 72 of return portion 67.

Once in return portions 67, cam followers 54 are permitted to move laterally with respect to body 12, but only in a single direction, which corresponds to the advancing direction of piston 30. Accordingly, base 46 is also permitted to move in the advancing direction by the user. The profile of return portion 67 is such that it ends in the lower portion 69 of the other actuating portion 64 of cam track 56. Accordingly, as the user rotates base 46 in the advancing direction to provide a dose of the composition, the button is urged outward by the movement of cam follower 54 within cam track thereby returning pushbutton 52 to its original position when cam followers snap over ramps 77 so as to be positioned in lower end 68 of actuation portion 64.

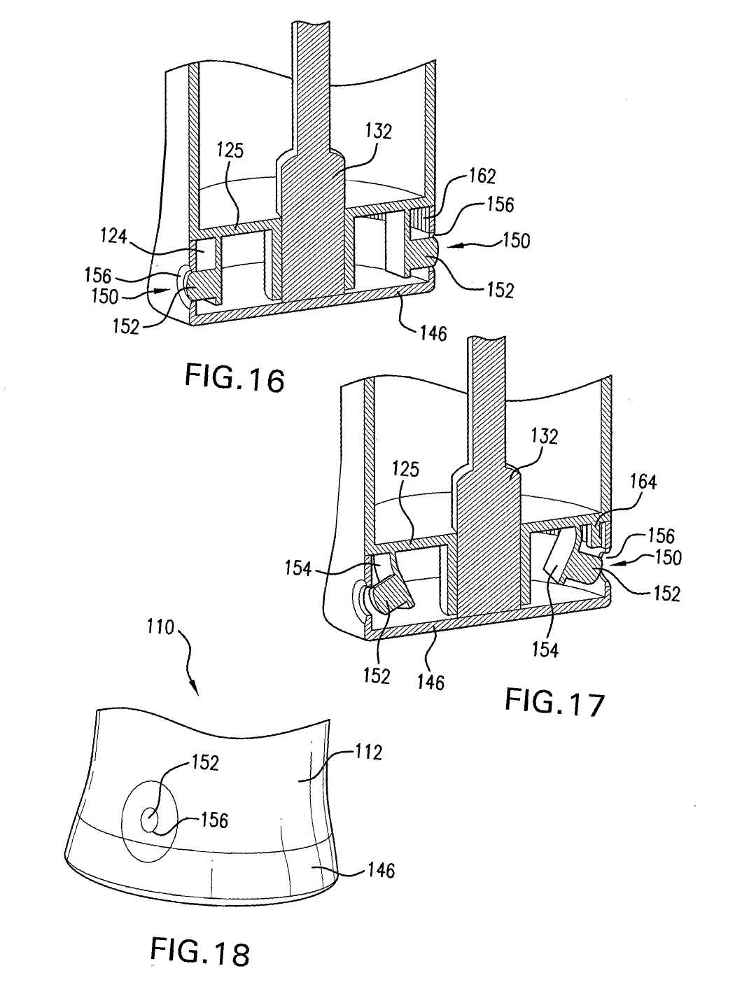

In an alternative embodiment shown in FIGS. 16 and 17, pushbutton 152 is affixed to a tab 154 that projects from the outside surface of proximal wall 125. Pushbutton 152 is arranged to fit into and extend through hole 156 formed in base 146 when the locking mechanism is in the active state (FIG. 16). When locking mechanism 150 is in the inactive state (FIG. 17), tabs 154 are flexed inward and pushbuttons 152 are spaced inwardly of holes 156 so that base 146 is free to rotate with respect to body 112. In order to carry out dosing, which is effected by twisting of base 146 relative to body 112, locking mechanism 150 must first be released. This is done by depressing pushbuttons to change locking mechanism to the inactive state. Once rotation of base 146 causes hole 156 to move out of alignment with pushbutton 152 (FIG. 17), the locking mechanism is held in the inactive state until hole 156 moves back into alignment with pushbutton 152 (FIG. 16). While in the inactive state, tab 154 is flexed causing pushbutton 152 to exert a force against base 146. Once hole 156 becomes re-aligned with pushbutton 152, which preferably takes place at the end of the dosing action, tab 154 relaxes and causes pushbutton 152 to extend through hole 156, thereby returning the locking mechanism to the active state. To improve ease of use, the free end of pushbutton 152 is preferably curved to provide a leading edge that is spaced inwardly of the outside end of pushbutton 152. This reduces the distance at which pushbutton must be depressed to allow the user to turn base 146 and improves the ease of use.

As shown in FIGS. 16 and 17, the locking mechanism can include two sets of pushbuttons 152, tabs 154, and holes 156, each set being located on opposite sides of base 146. Such an arrangement is preferred when the dosing involves turning base 146 through an angle of 180° because it allows for automatic return of the locking mechanism to the active state after each dose. It is noted that the locking mechanism 150 in such an embodiment does not have to include two pushbuttons 152 and two holes 156, but rather can include a single hole and two pushbuttons, or two holes and a single pushbutton. Two pushbuttons 152 and holes 156, however, can lead to a more robust mechanism. Furthermore, in an alternative arrangement shown in FIG. 18, the pushbutton 152 and tab 154 can be affixed to base 146, and hole 156 can be formed in body 112. The operation of this locking mechanism 150 is substantially the same as discussed with reference to FIGS. 16 and 17. In an embodiment of dispenser 110 in which dosing is carried out by a 120° turn, three pushbuttons 152 affixed to three tabs 154 can interact with a single hole 156, for example, to provide for automatic re-locking after each dose. Other locking mechanisms and arrangements according to the principles discussed above are contemplated for other dosing angles. In a further embodiment, the locking mechanism can additionally or alternatively require a second unlocking action, such as movement of base 146 toward body 112.

In these embodiments, dispenser 110 also preferably includes an anti-backup mechanism. As shown in FIG. 16, such an anti-backup mechanism can include a plurality of ratchet teeth 162 formed around the circumference of the inside surface of base 146. The anti-backup mechanism can also include a pawl 164 that projects downwardly from body 112 that engages at least one of the ratchet teeth 162. Preferably, the ratchet teeth 162 and the pawl 164 interact to permit rotation of the base 146 relative to the body 112 in the advancing direction, but prevent rotation of the base 146 relative to the body 112 in a direction opposite to the advancing direction. This helps to prevent air from being drawn back into supply chamber 120, and can reduce leaking or loss of pressure in dispenser 110. It also can improve dosing accuracy and provide audible and tactile feedback during dosing.

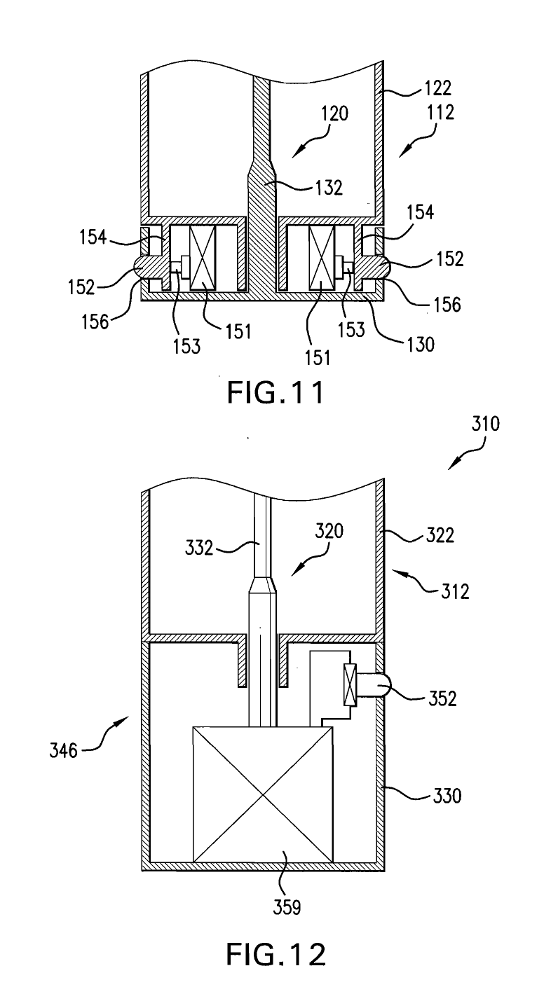

In one embodiment shown in FIG. 11, the locking mechanism can also include a timer circuit 151 that is connected to a power source and configured to control an actuator 153 arranged to extend and contact the inner surface of tab 154 in order to lock and to retract away from tab 54 in order to unlock the movement of pushbutton 152. Timer circuit 151 can be programmed to prevent the user of the device from being able to dispense more than a set number of doses within a predetermined time period. For example, the timer circuit can be programmed to prevent more than a single dose from being administered within a 24-hour period.

Alternative arrangements for the advancement mechanism are also contemplated. For instance, dosing can further require a dosing button to be depressed subsequent to twisting to pressurize active volume 20a. The dosing button can be associated with the application surface 70 such that the dose is released when the application surface is brought into contact with the skin of the patient. Furthermore, the dosing dial can be located on the distal end of dispenser 10 or the dosing button can be located on the proximal end of dispenser 10. Further, the dosing dial and the dosing button can be formed in a single element that can carry out both functions based on different actions.

As shown in FIG. 12, an embodiment of dispenser 310 can include a motor 359 that is affixed within base 346 that is non-rotatably affixed to body 312. Motor 359 is electronically connected to a power source, and is further connected to a switch 352 that allows a user of the device to turn motor 359 on and off. Motor 359 is affixed at the output end thereof to rod 332 such that turning the motor 359 on causes rotation of the rod 332 in an advancing direction to cause the composition to be dispensed. Dispenser 310 can also have a timer circuit included therein that can be programmed to prevent the user of the device from being able to dispense more than a set number of doses within a predetermined time period. For example, the timer circuit can be programmed to prevent more than a single dose from being administered within a 24-hour period.

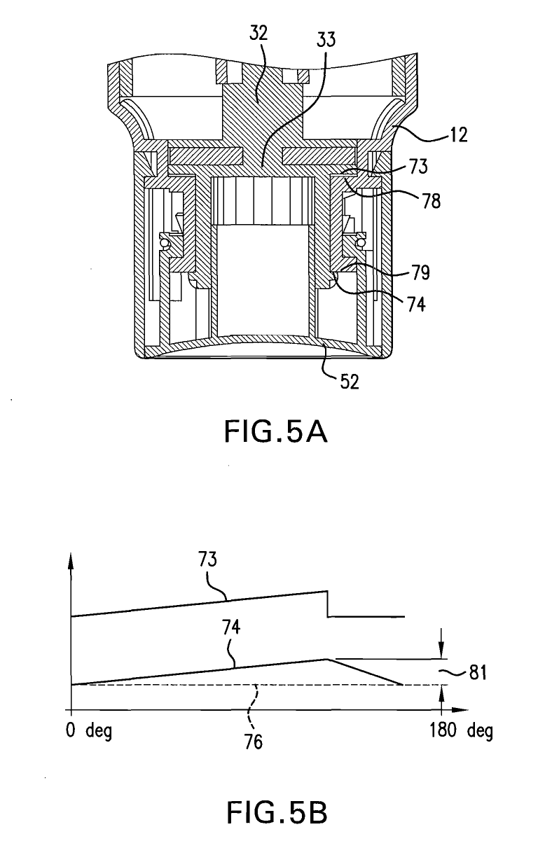

In an embodiment shown in FIGS. 5A and 5B, rod 32 is moveably affixed to base 46 such that it is moveable in a direction along the central axis of rod 32. In this arrangement rod 32 is configured to attach to base 46 such that it is moveable along the central axis only and prevented from twisting with respect to base 46 so that turning of base 46 results in turning of rod 32. In the particular arrangement shown in FIG. 5A, rod 32 is moveably affixed to pushbutton 52, which is, in turn, affixed to base 46 as discussed above, although other arrangements are possible. The moveable arrangement of rod 32 to base 46 is utilized to impart a reciprocating action on rod 32 and thereby piston 30 during the dosing action of device 10. As shown in FIGS. 5A and 5B, body 12 includes an upper cam 78 and a lower cam 79 and rod 32 includes a lower portion 33 having an upper cam follower 73 and a lower cam follower 74. Upper 78 and lower 79 cams are shown in a 2-dimensional representation thereof in FIG. 5B in which the surface of lower cam 79 faces downward and the surface of upper cam 78 faces upward relative to the depiction of FIG. 5B. Cams 78,79 are configured so as to raise rod 32 by a predetermined height 81 above its initial position 76 over a majority of the dosing motion (shown exemplary as 180° in FIG. 5B) and then to return rod 32 to its initial position 76 by the end of the dosing motion.

The effect of this arrangement is to provide an uneven pressure level during dosing such that the desired dose is expelled before the end of the dosing motion because of increased pressure and then to reduce the pressure within the supply chamber 20 as the dosing motion is completed. This pressure reduction between dosing motions is preferably enough to substantially eliminate, or relieve, the pressure of the liquid medicament contained in the supply chamber 20 while the device 10 is being stored. Relieving the pressure of the medicament between uses reduces the level of strain that device 10 is subjected to over its life and reduces the risk of leaking during storage. The pressure reduction can also allow for any valve system used in connection with opening 86 to use a lower minimum opening pressure, making the device 10 easier to use, and in particular making the dosing movement easier to carry out. Exemplary valve arrangements are discussed below.

Returning to FIGS. 1 and 2, distal wall 24 of supply chamber 20 is preferably generally conical, narrowing in width in the distal direction. The conical shape of distal wall 24 aids in filling supply chamber 20 with the composition in that it allows for the escape of air bubbles that can otherwise become trapped within supply chamber 20. Such a structure can be useful in an embodiment in which distal end wall 24 is formed on a separate end cap 66 that can be removed from the remainder of body 12. In such an embodiment, end cap 66 allows for filling of supply chamber 20 below the upper rim 41 formed when end cap 66 is removed. The end cap 66 can then be installed and the resulting headspace purged, for example by distal movement of piston 30. Without the conical shape of end cap 66, air might become trapped, or if the supply chamber 20 is filled in a vacuum, voids could be formed that can draw in air when the vacuum is released. Preferably, the upper surface of piston 30 has a shape that substantially fits within distal wall to allow substantially all of the composition to be expelled from supply chamber 20. Preferably, up to 98% of the composition can be expelled from supply chamber 20 by complete advancement of piston 30 in supply chamber 20. Preferably, end cap 66 attaches to the remainder of body 12 by a snap fit, although other attachments, such as a screw fit, are contemplated.

An application surface 71 is formed in the central portion of distal surface 70. Conduit 26 is preferably located near the center of application surface 71 and provides fluid communication between application surface 71 and supply chamber 20, such that an amount of the composition expelled during dosing flows onto application surface 71. Applicator surface 71 is preferably concave in shape and is sufficiently sized such that the predetermined amount of the composition expelled from supply chamber 20 during dosing can be held in the depression formed by applicator surface 71 when dispenser 10 is in the upright position. Accordingly, the volume of the depression formed by applicator surface 71 that is preferably enclosed by the upper rim 42 thereof is preferably greater than or equal to the volume of the predetermined dose. Furthermore, applicator surface 71 is preferably shaped so that the patient's skin can contact substantially the entire concave applicator surface 71 when the composition is being applied to the skin.

The maximum depth of the application surface 71 below rim 42 may correspond to the area of the application surface 71 defined within the rim 42 and may additionally or alternatively vary with the length of a minor axis of application surface 71. In the embodiment shown in FIG. 2, rim 42 is substantially planar, and application surface preferably has an area of between 5 cm² and 15 cm² and more preferably between about 8 cm² to 10 cm² and application surface 71 has a corresponding maximum depth of between 1 mm and 7 mm. In one embodiment, application surface 71 has a maximum depth of about 3 mm. Other embodiments are contemplated in which rim 42 has a substantially non-planar configuration, such as a saddle shape or the like.

Preferably, application surface 71 has a substantially oval or elliptical shape to substantially match the preferred elliptical shape of body 12. Preferably the elliptical shape of application surface 71 has a major axis that is at least about 1.1 times the length of a minor axis, and more preferably about 1.2 times the length of the minor axis. In some embodiments, the length of the major axis may be about twice that of the minor axis, but is preferably no more than 1.5 times the length of the minor axis. Further preferably the major axis of the elliptical shape has length of between about 35 mm and 45 mm. In one embodiment the major axis has a length of about 38 mm and the minor axis has a length of between 28 mm and 38 mm. In one embodiment, the major axis has a length of about 38 mm and the minor axis has a length of about 32 mm. In a further embodiment, the minor axis has a length of about 32 mm and the application surface 71 has a depth of between about 2 mm and 5 mm.

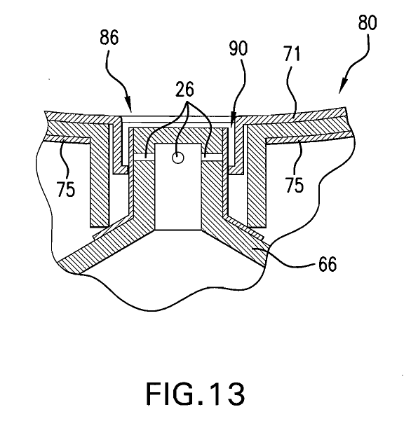

In a preferred embodiment, a secondary surface 43 is formed in distal surface 70 and surrounds the concave application surface 71. Secondary surface 43 is preferably sloped away from application surface 71 and is more preferably convex in shape. Secondary surface 43 may help to evenly apply the composition to the skin and to keep the composition near application surface 71. Furthermore, some medicaments are aided in absorption to the skin by application of heat, ultrasound (phonophoresis/sonophoresis-assisted dermal delivery) or of electric current (iontophoresis-assisted dermal delivery). In such instances, a heating element 75 (FIG. 13) or a source for generating heat, ultrasound or electric current can be disposed beneath distal surface 70 to provide heat, ultrasound, or electric current, respectively, therefor. A pressure switch can also be included in or below applicator surface to activate the heat, ultrasound or electric current source when distal surface 70 contacts the skin of the patient. Similarly, the applicator surface can include microneedles, microprojections or other types of abrasive features or materials, the function of which is to create painless, microscopic breakage of the stratum corneum and to thereby circumvent the barrier properties of this outermost layer of the skin. Such abrasive features can be formed integrally with application surface 71, or may be formed separately and then affixed to applicator surface, which may be done according to the general principles described in U.S. Pat. No. 6,136,008. Application surface 71 can contain as many as 300 microprojections per cm² of surface area. The individual microprojections or microneedles preferably each have a length of less than 200 μm.

As shown in FIGS. 1 and 2, distal surface 70, including application surface 71 and, preferably, secondary surface 43 can be formed on a removable applicator 80. Removable applicator 80 is preferably formed from PP and is configured to be disposable, thereby helping in cleanup of dispenser 10 after administration of a dose of the composition to the skin. Applicator 80 can be removed from body 12 after use and discarded without cleaning. This improves ease of use and reduces the risk of contamination, particularly when the dose is administered by someone other than the patient. Applicator 80 can be structured to include an application surface 71 having microprojections or microneedles formed thereon, as described above. Applicator 80 can be a thin-walled molding configured to fit onto the head of the dispenser with an opening 86 formed therein that is in fluid communication with conduit 26 to allow flow of the composition onto application surface 71. The thin design provides for reduced cost and allows for compact nesting in packaging of the components. For example, a stack height of about 5 mm per applicator in a stack of thirty applicators, or one-month's supply, would combine to a height of about 150 mm. Preferably a supply of applicators is provided with the dispenser that is equal to the number of predetermined doses within supply chamber 20 such that each applicator can be used in the application of a single dose and then discarded, a new applicator being used for each subsequent dose application.

Applicator 80 is preferably designed for optimized retention to the dispenser while allowing for easy removal when the application of the gel is complete. Applicator 80 engages the body 12 sufficiently to stay in place during vigorous application, but releases from body 12 when intended. To facilitate removal of applicator after dosing, a main flange 82 projects from the perimeter of applicator 80. The composition contained within dispenser may include hormones or other medicaments (for example medicaments used in treatment of: cancer; pain or affections of the Central Nervous System, such as Parkinson's or Alzheimer's Disease; depression and mood disorders; attention deficit and hyperactivity disorders; and the like) that would be harmful to such third-parties, should they contact the third party's skin. Therefore, in addition to providing assistance in removing applicator 80, the flange 82 preferably extends beyond the edge of distal surface 70 by a length sufficient to prevent a substantial portion of the composition present on the distal surface 70 from reaching outside edges of flange 82 so that the user can remove the applicator 80 without coming into contact with the composition. Applicator 80 can have a pair of flanges 82 projecting from opposite sides thereof or can include a single flange 82 formed around the entire perimeter of applicator 80 as shown in FIG. 1. Such a continuous flange 82 can extend by an additional length in specified gripping areas 84.

Further, a gripping surface can be provided, such as by gripping flanges 83, which are dimensioned and a configured to receive a user's fingers from underneath to remove the applicator 80 without touching the medicament. Gripping flanges 83 can be arched to facilitate grasping and can align with indentations 21 in body 12 to give extra room for a user to grasp the flanges 83. Preferably flanges 82 extend by at least one mm beyond the outer periphery of applicator 80. Further preferably, gripping flanges 83, at least in the intended gripping areas extend by between 3 mm and 6 mm and more preferably about 5 mm. Further, gripping flanges 83 can extend inwardly in addition to or alternatively to extending outwardly and may be present on the underside of a portion of applicator 80 that is wider than body 12, such as in the are of indentations 21 formed in body 12 or in an extended portion of applicator 80.

Applicator 80 can attach to body 12 by including a ridge 29 along the outside perimeter of body 12 near the distal end thereof along with a mating projection 85 directed inwardly from the perimeter of applicator 80 that together provide a snap-fit between the body 12 and the applicator 80. The projection or projections 85 of applicator 80 can be located in the area of gripping flanges 83 so that force on flange 82 causes deformation of applicator 80 which causes the projection to release from the ridge. Alternative attachment arrangements are possible according to similar principles. For example, body 12 can include a ledge or projection that is received within a groove or indentation formed on the inside surface of applicator 80. In addition or in the alternative, conduit 26 can be formed on a nozzle 27 and the inside of opening 86 can include an extension 88 that substantially mates with nozzle 27, forming a pressure fit therebetween. Extension 88 can also include an inwardly-turned end portion 89 that provides the primary contact interface between extension 88 and nozzle 27.

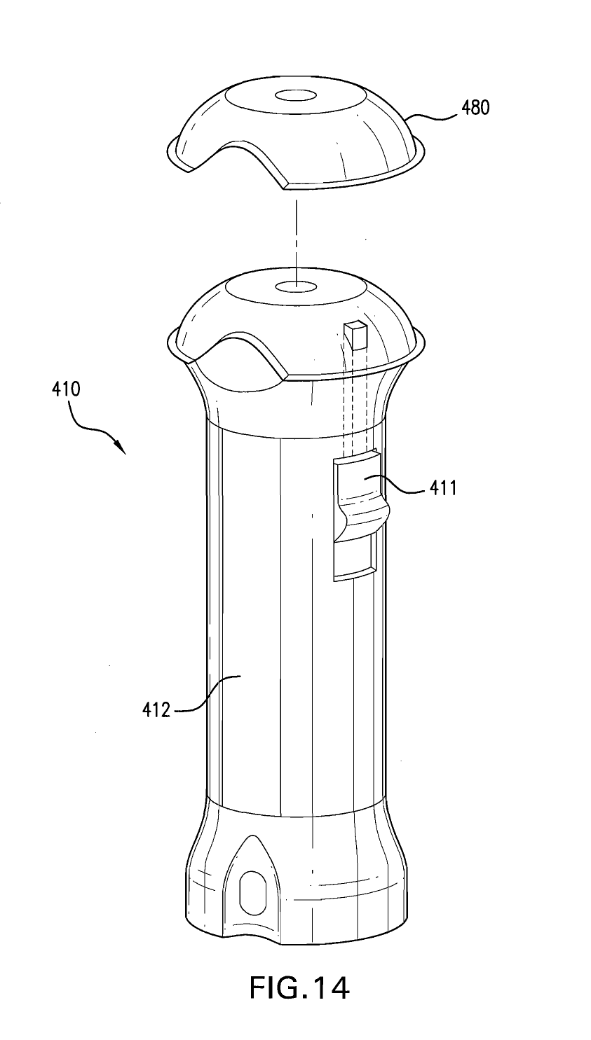

When applicator 80 is removed from body, end portion 89 preferably slides along nozzle 27 providing a wiping action, or squeegee effect, that removes at least a portion, and more preferably all or substantially all, of any residual the composition that is present on the surface of nozzle 27. In the preferred embodiment, the interface between the applicator 80 and the nozzle 27 wipes at least the radial surface of the nozzle 27. Additionally, as shown in FIG. 14, in an embodiment of dispenser 410 body 412 can include a slide member 411 that has an end that contacts the inner surface of applicator 480 such that movement of the sliding member 411 in a distal direction causes applicator 480 to become detached from body 412. Alternatively, extension 88 and nozzle 27 can include mating threaded portions to facilitate attachment therebetween. Preferably, the distal end of body 12 includes a support surface 28 that substantially mates with the inside surface of applicator 80 to provide support therefor, and preferably has a shape corresponding to the underside of the applicator 80.

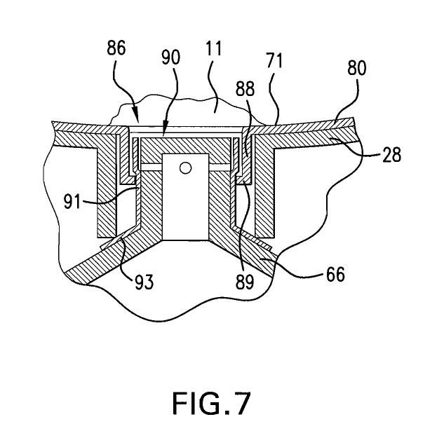

A valve 90 (FIG. 7) is preferably included in connection with conduit 26 to substantially seal supply chamber 20 from both unintended escape of materials out of supply chamber from ingress of contaminants from outside the supply chamber. Further, the composition intended to be applied to the skin using dispenser 10 may include volatile compounds that can evaporate either during storage or during use with prior containers. Evaporation of volatile compounds may adversely affect both dosing accuracy and medicament administration to the skin. Valve 90 may also be suitable to prevent evaporation of volatile compounds present in the composition. Generally, such volatile compounds will evaporate from the composition until an equilibrium vapor pressure is reached within the supply chamber. If valve 90 is unable to withstand the vapor pressure, the volatile compounds that have been released from the composition in gaseous form will pass out from valve, which will cause further evaporation of the volatile compound from within the composition and further leakage. Therefore, the valve 90 should be able to withstand the vapor pressure of the given volatile compound or compound contained therein for a reasonable temperature range. The enthalpy of vaporization for various volatile compounds which may be included in compositions administered using device 10 is shown in Table 1, which can be used to derive the appropriate pressure for valve 90 to withstand. Preferably, valve 90 is configured to function properly at temperatures of at least 0° C. Preferably valve 90 is further configured to function properly at temperatures of less than 45° C., and further preferably, valve 90 is configured to function at temperatures of between about 15 and 35° C. Further, contaminants intended to be kept out of supply chamber 20 include air.

TABLE 1
Enthalpy of vaporization of certain solvents

| Solvent | t_{b} | ΔᵥₐₚH (t_{b}) |
|---|---|---|
| Acetone | 56.0 | 31.3 |
| Ethanol | 78.3 | 38.6 |
| Propan-2-ol (isopropanol) | 82.3 | 39.9 |
| Propanol | 97.2 | 41.4 |
| Butan-2-ol | 99.5 | 40.8 |
| Butan-1-ol | 117.7 | 43.3 |
| Ethylene glycol monomethyl ether | 124.1 | 37.5 |
| Ethylene glycol monoethyl ether | 135.0 | 39.2 |
| Ethylene glycol monopropyl ether | 149.8 | 41.4 |
| 1,2-Propylene glycol | 187.6 | 52.4 |
| Diethylene glycol monomethyl ether | 193.0 | 46.6 |
| Diethylene glycol monoethyl ether | 196.0 | 47.5 |
| 1,3-Propylene glycol | 214.4 | 57.9 |
| Glycerin | 290.0 | 61.0 |

Valve 90 is preferably self sealing; that is, while at rest, the valve should naturally keep the supply chamber sealed, preferably by forming an air tight seal. The use of a self-sealing valve can further allow a liquid to be stored in and dispensed using dispenser 10. Further, the valve should allow for the predetermined dose of the composition to escape from supply chamber 20 through conduit 26, when the device is activated, and automatically return to the sealed state once dosing is complete. In a preferred embodiment, the valve includes a deformable member 91 such that the valve can function without relative movement of mechanical parts. A valve 90 using a deformable member 91 should be structured such that pressure of the composition within supply chamber 20 provided by a dosing action, as described above, causes deformation of the deformable member, which allows the dose to be expelled. Once the expulsion of the dose of the composition relieves the pressure within supply chamber and on the deformable member, the deformable member returns to its original shape whereby conduit 26 is sealed. Valve 90 is preferably structured such that expulsion of the dose of the composition occurs at a predetermined pressure within the supply chamber. Such a predetermined pressure is at least 80 kPa, and is preferably less than about 120 kPa. In a preferred embodiment, valve 90 is structured to release the dose of the composition at a pressure of about 100 kPa.

The valve structure shown in FIGS. 2, 6, and 7 includes a deformable member 91, such as in the form of an elastomeric sleeve that fits around nozzle 27. Conduit 26 passes through nozzle 27 and is open to the lateral sides of the nozzle in at least one location therealong. Sleeve 91 is preferably affixed to nozzle 27 proximally of conduit 26 and extends distally to cover conduit 26. In the embodiment shown, sleeve includes a skirt 93 extending outwardly therefrom, which is held in place by a pressure fit between support surface 28 and end cap 66. In other embodiments sleeve 91 can be affixed to nozzle 27 by adhesives, welding or the like. As shown in FIG. 7, increased pressure of the composition against sleeve 91 causes sleeve 91 to deform outwardly away from conduit 26 to allow the composition to pass therethrough and out through opening 86 onto application surface 71. When the predetermined dose has been expelled through conduit 26, sleeve 91 returns to its original shape, forming a seal over conduit 26.

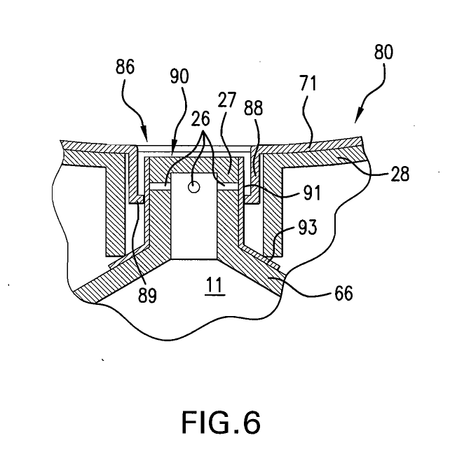

A valve structure including an elastomeric sleeve 91 can interact with a removable applicator 80 as shown in FIG. 6 to remove residual the composition that can collect between sleeve 91 and nozzle 27 or on the outside of sleeve 91. Applicator 80 is preferably constructed such that the end portion 89 contacts the outside surface of sleeve 91, such that when applicator 80 is removed from body 12, the end portion 89 provides a wiping action to substantially remove the composition from the outside surface of sleeve 91, which can prevent clogging of nozzle 27 or valve 90. Additionally, the pressure applied by the wiping action can force any of the composition held between sleeve 91 and nozzle 27 out from the distal end thereof.

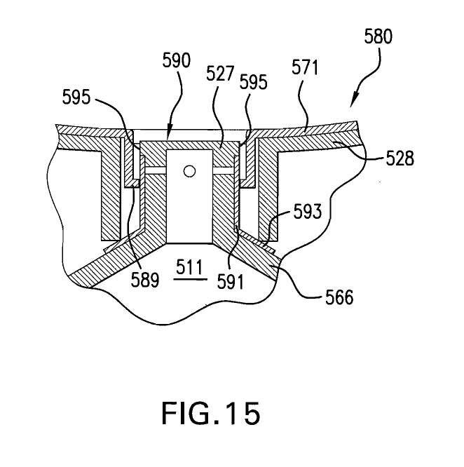

In the embodiment shown in FIG. 15, nozzle 527 is preferably constructed such that the composition is dispensed laterally, from the side of the nozzle 527. Such an arrangement results in end portion 589 passing over the fluid outlet formed in nozzle 527 so as to provide a wiping action therefor. Such a wiping action prevents residual composition from drying over the outlet of nozzle 527, which may lead to clogging. In this embodiment, elastomeric sleeve 591 ends terminates below the top of nozzle 527, preferably adjacent thereto and in abutment therewith, and nozzle 527 includes an outer flange 595 that directs the flow of composition radially to provide the preferred location for the outlet 529 of nozzle 527. The preferred thickness of the sleeve 591 is selected to provide a generally smooth transition with the lateral surface of the flange 595 to allow the end portion 589 to wipe any or most of the composition that may remain exposed at the outlet. While in this embodiment, the outlet is in the shape of a ring surrounding the nozzle 527, other embodiments have differently shaped or discontinuous outlets.

Alternatively, extension 88 can be structured so as to fit between nozzle 27 and sleeve 91. This fit can be facilitated by tapering nozzle 27 at the distal end thereof or by flaring sleeve 91 at the distal end thereof. This arrangement blocks the composition from reaching the outside of sleeve 91 and provides a wiping action between wiping portion 89 and the surface of nozzle 27 when applicator 80 is removed from body 12. The wiping portion 89 is preferably enlarge radially compared to the adjacent extension 88 to increase the pressure against the nozzle 27 received therein and improve the wiping action.

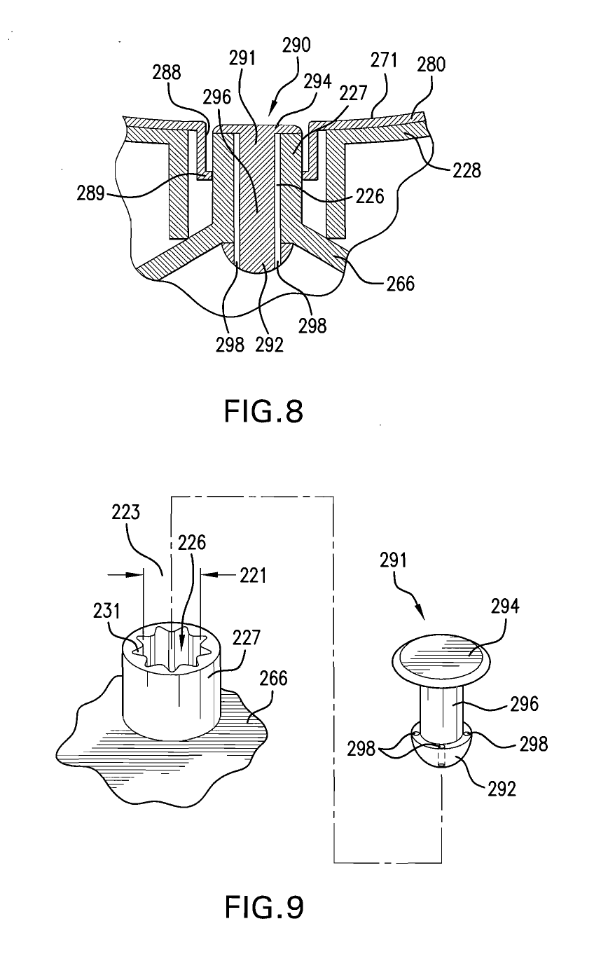

An alternative embodiment of a valve 290 including a deformable member 291, shown in FIGS. 8 and 9, is in the form of an umbrella valve, and includes a deformable member 291 in the general shape of an umbrella. Umbrella 291 includes proximal end 292, a distal end 294, and a midsection 296 that joins proximal end 292 and distal end 294. Umbrella 291 is preferably formed from an elastomeric material such as TPE or silicon rubber. In general, umbrella 291 should be formed from a material that exhibits acceptable resistance to the chemicals in the composition and should be appropriate for skin contact, as well as being able to carry out the necessary deformation and providing the desired seal. Conduit 226 is preferably shaped such that midsection 296 can pass therethrough so as to help retain umbrella 291 therein. Proximal and distal portions 292, 294 are preferably shaped so as to be wider than conduit 226 such that umbrella 291 is secured within conduit 226. The length of midsection 296 should be such that midsection 296 is tensioned slightly when umbrella 291 is in place within conduit 226 so that distal portion 294 is held against the end of nozzle 227 to provide the desired seal. In one embodiment, midsection 296 has a diameter that is slightly less than the diameter of conduit 226 to allow escape of composition therebetween.

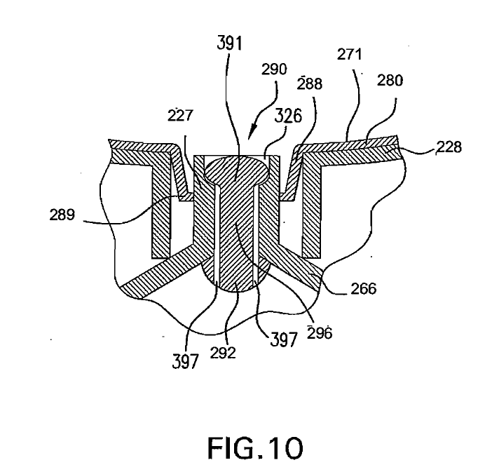

In the embodiment shown in FIG. 8 midsection 296 has a diameter which substantially matches an inner diameter 221 of conduit 226 or is naturally slightly larger. Conduit 226 includes channels 231 formed therein to allow material to flow therethrough toward distal portion 294. In a further alternative, midsection has a diameter that substantially fits within conduit 226 and has channels formed therein to permit the flow of the composition (see channels 397 in FIG. 10). Proximal portion 292 includes at least one passageway, which can be slots or holes 298 formed therein for example, to allow the composition to pass therethrough and into conduit 226. When the proscribed dosing operation causes the pressure of the composition to increase within supply chamber, the fluid communication provided through proximal portion 292 and between midsection 296 and conduit 226 causes the composition to apply pressure to the inside surface of distal portion 294, which in turn causes deformation of umbrella 291, and in particular distal portion 294, away from the end of nozzle 227, which distal portion 294 overlies. This deformation permits escape of the composition through conduit 226 and onto application surface 271. In an alternative arrangement shown in FIG. 10, conduit 326 can be structured to allow a modified umbrella valve 391 to fit therein. This type of umbrella valve 391 functions by deforming primarily inwardly under pressure from the composition.

Dispenser 10 can also include a dosing indicator as shown in FIGS. 1 and 2. A dosing indicator can include an arrangement that indicates the position of piston 30 within supply chamber 20. Preferably, a portion of piston 30 is visible through window 15 formed in body 12. In the embodiment depicted, the portion of piston 30 that directly contacts inside surface of supply chamber 20, which is preferably an o-ring 38 but can also be a lip, is visible through window 15. Window 15 can be a separately molded or assembled portion of clear plastic that is affixed to body 12. For sealing purposes, however, the entire body 12 is preferably formed from transparent or semi-transparent material to allow visualization of at least a portion of piston 30. It is not necessary that window 15 be completely transparent, it must only be transparent enough to visually discern a portion of piston 30. In an embodiment including a transparent body 12, a layer or film such as a label 17, which may be in the form of a self-adhesive sticker, is applied to the outside surface of body 12, which can be transparent in the desired area of window 15 or can include a cutout that fits around the desired location for window 15. Such a label 17 can also aide in sealing any seams in body 12 due to assembly (of end cap 66 onto body 12, for example) or molding. Label 17 can further be structured to provide a barrier for body 12. Exposure to UV-light can be detrimental to some active ingredients that may be delivered by dispenser 10. In such cases, and in particular when body 12 is formed of a transparent material, label 17 can be formed from a material that blocks UV-light or a material that has a UV-light-blocking coating applied thereto. In doing so, application of label 17 provides a barrier for blocking UV-light from passing into supply chamber 20. Additionally or alternatively, label 17 can be formed of a material that can prevent leaching of the composition into or through side wall or that can prevent contaminants from passing through side wall 22 from the outside of device 10 and into supply chamber 20. A label forming such a barrier layer can be applied to the inside of side wall 22, and may be visible from the outside of a transparent body 12.

Preferably, label 17 includes an indicia which can be in the form of numbering which provides visual indicia relating to dosing, preferably to the number of remaining doses within supply chamber 20. The number of remaining doses is determined by the position of piston 30. Preferably the numbers correspond to arrows 19, which line up with o-ring 38 and can be printed on label 17 or integrally molded into the outside surface of body 12. Integral formation of arrows 19 can reduce impact of label position tolerances. Further, the indicia can also relate to days of the week on which dose is to be administered and can include a removable or additional portion to properly indicate the days, which can be used in instances wherein dosing does not begin on a predetermined day. Label 19 can also include drug information required with certain compositions and in certain applications and can further include instructions on use. A “red zone” for last few doses can be indicated on label 19. Preferably, label 19 both functions as a barrier layer and includes dosing indicia.

When administering doses of composition, it is preferred that the composition be applied in a thin layer. To accomplish such administration, it is preferable to apply the desired dosage, for example per day, in multiple, smaller doses to the same area at spaced-apart times throughout the day, or to apply multiple, smaller doses to different areas of the body at a single time. In certain applications, a combination of spaced-apart and multiple-site dosing may be used. For example, a per-day dose of 5 g can be administered by applying thin layers of about 1.6 g of the composition to a single area of the body at three spaced-apart times during each day for the duration of treatment. Further, a 3 g dose of the composition can be applied as 1.5 g sub-doses of the composition at substantially the same time to two different areas of the patient's body, for example both of the patient's forearms.

Additionally, in order to further preserve the composition held within supply chamber 20 of the device during shipping or storage thereof, the device, once filled, can be placed in a sealable bag. The sealable bag preferably provides a barrier against contaminants to prevent such contaminants from coming into contact with the dispenser. Preferably, such a sealable bag is vacuum sealed or may contain nitrogen or other chemicals that can absorb any oxygen present therein to prevent any oxygen from reaching supply chamber and contaminating the composition.

### EXAMPLES

### Example 1

The supply chamber of a dispenser as disclosed herein is provided with approximately 100 mL of a transdermal gel formulation including oxybutynin according to examples 1 or 2 of U.S. patent application Ser. No. 11/120,306. This amount represents about 60 doses of 1.4 g of gel or about 30 doses of 2.8 g of gel. Applied once daily, the dispenser provides about 60 days (one dose of 1.4 g of gel per day), or about 30 days (one dose of 2.8 g of gel per day) of treatment of overactive bladder and urge incontinence for a patient.

### Example 2

The supply chamber of a dispenser as disclosed herein is provided with approximately 60 mL of a transdermal gel formulation including a combination of an estrogen and a progestin according to examples 1, 4, 13, 14, 15, 16, or 17 of U.S. Pat. No. 5,891,462. This amount represents about 60 doses of 0.75 g of gel or about 30 doses of 2.5 g of gel. Applied once daily, the dispenser provides about 60 days (one dose of 0.75 g of gel per day) or about 30 days (one dose of 1.5 g of gel per day) of hormonal replacement therapy treatment or contraception for the patient.

### Example 3

The supply chamber of a dispenser as disclosed herein is provided with approximately 90 mL of a transdermal gel formulation including estradiol according to Table 1 of Example 2 of U.S. Pat. No. 7,198,801. This amount represents about 120 doses of 0.6 g of gel, about 60 doses of 1.25 g of gel or about 30 doses of 2.5 g of gel. Applied once daily, the dispenser provides about 120 days (one dose of 0.6 g of gel per day), about 60 days (one dose of 1.25 g of gel per day) or about 30 days (one dose of 2.5 g of gel per day) of treatment of moderate-to-severe hot flashes associated with menopause in a female patient.

### Example 4

The supply chamber of a dispenser as disclosed herein is provided with approximately 50 mL of a transdermal gel formulation including testosterone according to Table 2 of U.S. Pat. No. 7,198,801. This amount represents about 120 doses of 0.2 g of gel, about 60 doses of 0.4 g of gel or about 30 doses of 0.8 g of gel. Applied once daily, the dispenser provides about 120 days (one dose of 0.2 g of gel per day), about 60 days (one dose of 0.4 g of gel per day) or about 30 days (one dose of 0.8 g of gel per day) of treatment of moderate-to-severe hot flashes associated with menopause in a female patient.

### Example 5

The supply chamber of a dispenser as disclosed herein is provided with approximately 100 mL of a transdermal gel formulation including alprazolam according to example 23 of U.S. Pat. No. 7,214,381. This amount represents about 90 doses of 1.0 g of gel, about 45 doses of 2.0 g of gel or about 30 doses of 3.0 g of gel. Applied once daily, the dispenser provides about 90 days (one dose of 1.0 g of gel per day), about 45 days (one dose of 2.0 g of gel per day) or about 30 days (one dose of 3.0 g of gel per day) of treatment of panic attacks.

### Example 6

The supply chamber of a dispenser as disclosed herein is provided with approximately 100 mL of a transdermal gel formulation including amlodipine according to example 38 of U.S. Pat. No. 7,214,381. This amount represents about 30 doses of 3.0 g of gel. Applied once daily, the dispenser provides about 30 days (one dose of 3.0 g of gel per day) of treatment of hypertension.

### Example 7

The supply chamber of a dispenser as disclosed herein is provided with approximately 85 mL of a transdermal gel formulation including L-thyroxine according to example 31 of U.S. Pat. No. 7,214,381. This amount represents about 30 doses of 2.5 g of gel. Applied once daily, the dispenser provides about 30 days (one dose of 2.5 g of gel per day) of treatment of hypothyroidism.

### Example 8

The supply chamber of a dispenser as disclosed herein is provided with approximately 85 mL of a transdermal gel formulation including testosterone according to example 5 of U.S. patent application Ser. No. 11/371,042. This amount represents about 30 doses of 2.5 g of gel. Applied once daily, the dispenser provides about 30 days (one dose of 2.5 g of gel per day) of treatment of male hypogonadism.

### Example 9

The supply chamber of a dispenser as disclosed herein is provided with approximately 70 mL of a transdermal gel formulation including fentanyl according to example 22 of U.S. patent application Ser. No. 11/371,042. This amount represents about 30 doses of 2.0 g of gel. Applied once daily, the dispenser provides about 30 days (one dose of 2.0 g of gel per day) of treatment of pain associated with cancer.

### Example 10

The supply chamber of a dispenser as disclosed herein is provided with approximately 50 mL of a transdermal gel formulation including pramipexole according to example 17 of U.S. patent application Ser. No. 11/770,194. This amount represents about 90 doses of 0.5 g of gel. Applied once daily, the dispenser provides about 90 days (one dose of 0.5 g of gel per day) of treatment of Parkinson's Disease.

### Example 11

The supply chamber of a dispenser as disclosed herein is provided with approximately 150 mL of a transdermal gel formulation including nicotine according to example 17 of U.S. patent application Ser. No. 11/492,568. This amount represents about 90 doses of 1.5 g of gel or about 45 doses of 3.0 g of gel. Applied once daily, the dispenser provides about 90 days (one dose of 1.5 g of gel per day) or about 45 days (one dose of 3.0 g of gel per day) of treatment relating to smoking cessation.

### Example 12

The supply chamber of a dispenser as disclosed herein is provided with approximately 45 mL of a transdermal gel formulation including selegiline according to example F of U.S. patent application Ser. No. 11/755,923. This amount represents about 60 doses of 0.5 g of gel or about 30 doses of 1.0 g of gel. Applied once daily, the dispenser provides about 60 days (one dose of 0.5 g of gel per day) or about 30 days (one dose of 1.0 g of gel per day) of treatment of Parkinson's Disease.

### Example 13

The supply chamber of a dispenser as disclosed herein is provided with approximately 150 mL of a transdermal gel formulation including pergolide according to example G of U.S. patent application Ser. No. 11/755,923. This amount represents about 30 doses of 5.0 g of gel. Applied once daily, the dispenser provides about 1 month of treatment of Parkinson's Disease.

### Example 14

The supply chamber of a dispenser as disclosed herein is provided with approximately 100 mL of a transdermal gel formulation including rivastigmine according to example C of U.S. patent application Ser. No. 11/755,923. This amount represents about 90 doses of 1.0 g of gel, about 60 doses of 2.0 g of gel or about 30 doses of 3.0 g of gel. Applied once daily, the dispenser provides about 90 days (one dose of 1.0 g of gel per day), about 60 days (one dose of 2.0 g of gel per day) or about 30 days (one dose of 3.0 g of gel per day) of treatment of Alzheimer's Disease.

### Example 15

The supply chamber of a dispenser as disclosed herein is provided with approximately 35 mL of a transdermal gel formulation including granisetron according to example J of U.S. patent application Ser. No. 11/755,923. This amount represents about 30 doses of 1.0 g of gel or about 15 doses of 2.0 g of gel. Applied once daily, the dispenser provides about 30 days (one dose of 1.0 g of gel per day) or about 15 days (one dose of 2.0 g of gel per day) of treatment of nausea.

The preceding examples illustrate preferred gels and preferred drug concentrations in said gels for use in the dispensers of the invention, but any of the other gels mentioned in the foregoing patents and patent applications may be used in these dispensers if desired. Accordingly, each patent and patent application mentioned herein is expressly incorporated by reference for its disclosure of the gels described therein, and the present invention covers the combination of each such gel in a dispenser as described herein.

While it is apparent that the illustrative embodiments of the invention herein disclosed fulfill the objectives stated above, it will be appreciated that numerous modifications and other embodiments may be devised by those skilled in the art. Therefore, it will be understood that the appended claims are intended to cover all such modifications and embodiments which come within the scope of the present invention.

What is claimed is:

1. A dermal flowable-composition dispenser comprising:
a body defining a supply chamber therein for retaining a supply of the composition;
an application portion having an application surface; and
a conduit connecting the supply chamber with the application surface for facilitating the reproducible delivery of a predetermined dose of the composition from the supply chamber to the application surface;
wherein the application portion has a front side and is disposed externally and facing away from the supply chamber for holding the predetermined dose of the composition for application to a patient's skin;
wherein the application surface includes a concave application surface and a front side having an upper rim enclosing the application surface and forming a depression that has a volume that is greater than or equal to the volume of the predetermine dose;
wherein the rim is substantially planar, the application surface has a surface area of between 5 cm² and 15 cm² with a maximum depth of between 1 and 7 mm; and
wherein the application surface is configured and dimensioned such it can receive the patient's skin to allow for removal of the predetermined dose of the composition, and wherein the concave application surface is shaped such that the entire application surface contacts the patient's skin when the application surface is pressed thereagainst.

2. The dispenser of claim 1, further comprising a discharge opening associated with the application surface and a one-way, self-sealing valve associated with the discharge opening, wherein the valve is configured to permit the dose of the composition to be expelled through the opening, otherwise keeping the opening substantially sealed so as to contain any vapor evaporated from volatile components of the composition within the body.

3. The dispenser of claim 2, wherein the application portion comprises an applicator that is removably affixed to the body and further comprising an applicator removal mechanism comprising an outwardly projecting flange disposed near an outer edge of the applicator configured to assist a user of the dispenser in removing the applicator from the body without contacting the application surface by receiving a user's finger therebeneath to remove the applicator from the body.

4. The dispenser of claim 3, wherein the conduit is disposed generally centrally in the application surface and comprises a nozzle, and the one-way, self-sealing valve comprises a sealing extension extending away from the application surface and defining an opening configured to receive the nozzle in fluid-tight association therewith to retain the composition delivered from the nozzle, and wherein the sealing extension includes an inwardly-turned portion configured and disposed for abutting and sealing against the nozzle and configured for wiping the composition from the nozzle when the applicator is removed from the body.

5. The dispenser of claim 2, wherein the one-way, self-sealing valve includes an resiliently deformable member secured to the discharge opening such that pressure of the composition against the resiliently deformable member causes deformation thereof sufficient to allow the composition to be expelled through the opening and such that, upon release of pressure against the resiliently deformable member, the resiliently deformable member returns to a sealing state.

6. The dispenser of claim 5, wherein the resiliently deformable member includes a proximal portion and a distal portion that covers the opening, and wherein the composition traveling through the opening causes outward deformation of the distal portion of the resiliently deformable member away from the opening to allow expulsion of the dose of the composition.

7. The dispenser of claim 6, wherein the proximal portion of the resiliently deformable member has a diameter that is larger than a diameter of the discharge opening and includes at least one channel formed therein to allow the composition to flow to the opening.

8. The dispenser of claim 1, further including an electronic delivery-assisting element associated with the application portion for providing a stimulus to the patient's skin.

9. The dispenser of claim 1 which further comprises a driving mechanism having a piston arranged within the body such that motion of the piston toward the discharge opening increases pressure within the supply chamber to cause a predetermined dose of the composition to be expelled through the conduit, valve and discharge opening;
wherein the piston includes a self-threading element, and the driving mechanism includes a rod engaged with the self-threading element such that relative rotation between the rod and the self-threading element cause the self-threading element to produce threads on the rod for advancing the piston along the rod while retaining a sealed association between the self-threading element and the rod, wherein the body has a non-circular outer profile defining a supply chamber for retaining a supply of the composition and having a central axis therein;
and the piston is slidably arranged within the supply chamber and having an aperture formed therethrough, the rod of the driving mechanism passes through the aperture and is arranged such that rotation of the rod causes linear motion of the piston along the central axis of the supply chamber, and the driving mechanism includes a base defining an outer, non-circular profile that substantially matches the non-circular profile outer of the body and is affixed to the rod such that a user can cause advancement of the piston by rotating the base; wherein the advancement mechanism is arranged such that a predetermined dose of the composition is supplied through the conduit upon rotation of the base through an angle that is about equal to a predetermined integer factor of 360°, and such that the non-circular profile of the base aligns with the non-circular outer profile of the body upon rotation of the base through the predetermined angle, but is out of alignment therewith at other rotational positions.

10. The dispenser of claim 9, which further comprises a locking mechanism disposed between the body and the base, wherein the locking mechanism is moveable between an active state and an inactive state such that motion of the base member relative to the body is restricted when the locking member is in an active state and the base is free to be rotated in an advancing direction when the locking member is in an inactive state.

11. The dispenser of claim 10, wherein the body includes a continuous cam track having one or more substantially vertical actuation portions and one or more gradually sloping return portions, and the locking member includes one or more pushbuttons configured for actuation by a user with the pushbutton(s) disposed within the dispenser base and including one or more cam followers that are urged into the cam track by a spring, wherein actuation of the pushbutton(s) changes the locking mechanism from the active state to the inactive state by lifting the cam followers through the actuation portions of the cam track into the return portions of the cam track.

12. The dispenser of claim 10 further comprising an advancement mechanism that, first, advances the piston to expel the predetermined dose of the composition, and second, retracts the piston so as to reduce pressure in the supply chamber, wherein the piston is advanced relatively gradually but retracted relatively rapidly wherein the mechanism permits rotation of the base member relative to the body in an advancing direction, but prevents rotation of the base member in a retracting direction.

13. The dispenser of claim 9, further comprising a layer affixed to a surface of the body and configured as a barrier to protect the composition, wherein the layer and the body include co-aligned transparent portions extending longitudinally therealong such that a portion of the piston is visible therethrough, and wherein the layer further includes a plurality of visual indicia associated with the transparent portions for indicating a number of doses of the composition dispensed or remaining in the supply chamber, with the layer optionally having UV-light blocking properties or chemical blocking properties.

14. The dispenser of claim 9, wherein the rod has one or more cam followers that engage one or more cams of the dispenser body so that relative rotation of the rod and the body causes a reciprocating axial motion of the rod during which the rod is axially advanced relatively gradually but axially retracted relatively rapidly, and the self-threading element is secured in an aperture of a piston and engages the rod so that rotation of the rod causes the nut to cut new threads in an advancing direction along the rod and optionally includes a cylindrical interior surface having two start threads formed therein on opposing sides thereof, wherein the start threads are each configured for forming a discrete thread set on the rod.

15. The dispenser of claim 9, further including a distal portion of the supply chamber having a tapered cross-section that narrows towards the discharge opening for facilitating filling of the composition into the supply chamber by allowing the escape of air bubbles, wherein the piston has a tapered cross-section that substantially matches the tapered cross-section of the supply chamber for maximizing the amount of composition that can be expelled.

16. The dispenser of claim 9, wherein the rod is moveably affixed to the base such that the rod is moveable with respect to the base in a direction of movement parallel to the central axis of the supply chamber, and wherein a portion of the rod interacts with a corresponding portion of the body to cause the rod to reciprocate along the direction of movement during rotation of the base through the predetermined angle.

17. The dispenser of claim 16, wherein the reciprocal movement of the rod during rotation of the base such that the pressure within the supply chamber is reduced after the predetermined dose of the composition is supplied.

18. The dispenser of claim 9, wherein the rod is an unthreaded cylinder and self-threading element is a nut that includes threads of left-hand configuration whereby clockwise rotation of rod causes motion of piston in the distal direction to cause self threading of the rod to form threads for advancement of the rod while providing sealing that reduces or prevents ingress of oxygen or other contaminants; leaking of the composition; loss of pressure within the active volume; or evaporation of volatile compounds within the composition.

19. The dispenser of claim 18, wherein the nut is a twin-start self-threading nut having two start threads formed therein such that two discrete thread sets are formed in the rod as the nut is advanced thereon to reduce the height of the nut overall size of the dispenser while providing balancing of forces applied to piston and rod during use thereof; and further comprising a seal around the perimeter of piston.
